SEQUENCE LISTING (8 sequences)

SEQ ID NO: 1 - protein, 495 aa, Artificial Sequence
  Amine oxidase mutant 1
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHIWAMSGYTYQGCRDCLISYKFKDGQSAFARRFWEEAAGTGRLGYVFGCPVRSVVNERDAARVTARDGREFAAKRLVCTIPLNVLSTIQFSPALSTERISAMQAGHVNMCTKVHAEVDNKDMRSWSGIAYPFNKLCVAIGDGTTPAGNTHLVCFGTDANHIQPDEDVRETLKAVGQLAPGTFGVKRLVFHNWVKDEFAKGARFFSRPGMVSECLQGLREKHRGVVFANSDWALGWRSFIDGAIEEGTRAARVVLEELGTKREVKARL

SEQ ID NO: 2 - protein, 495 aa, Artificial Sequence
  Amine oxidase mutant 2
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAMILCCSGGTLENSSFGEFLHWWAMSGYTYQGCVDCLTSYKFKDGQSAFAKKFWEEAAGTGRLGYVFGCPVRSVVNERDAARVTARDGREFAAKRLVCTIPLNVLSTIQFSPALSTERISAMQAGHVSMCTKVHAEVDNKDMRSWTGIAYPFNKLCYAIGDGTTPAGNTHLVCFGNSANHIQPDEDVRETLKAVGQLAPGTFGVKRLVFHNWVKDEFAKGAGFFSRPGMVSECLQGLREKHRGVVFANSDWALGWRSFIDGAIEEGTRAARVVLEELGTKREVKARL

SEQ ID NO: 3 - DNA, 1488 bp, Artificial Sequence
  Nucleotide sequence encoding amine oxidase mutant 1
atgacgagccgggatggataccagtggacgccggagacaggtttaacgcaaggcgtaccatcgcttggggtaatttcgccacctacaaacatcgaggacacagataaagacggtccttgggacgtgatagtgataggtggagggtactgcggattaactgccaccagagatctgactgtggcaggcttcaaaactcttctgttggaagctagagatcgtataggaggcagaagttggagctctaacatcgacggctatccctatgaaatgggcggaacttgggtacactggcatcaatctcacgtatggagagagattaccagatacaagatgcataatgccctgtctccatctttcaatttcagtcgcggggtcaatcactttcaattgagaactaaccccaccacaagcacgtacatgacccacgaggcggaagatgagctgttgcggagcgccttacataaatttacaaacgtggacggtacgaatgggcggacagtcttacctttcccgcacgatatgttttacgttcccgagtttcggaagtatgatgagatgagctattcagagcgtattgatcagattcgtgacgagctgtccttaaatgagcgctcatctctggaggcattcatccttttatgctctggcggaacgctggagaattctagcttcggagaatttctgcatatttgggccatgtccggttatacctaccaaggatgccgcgattgcttaatctcctataagttcaaagacggacagtcagcatttgctcggcgcttctgggaagaggcggcagggactgggagactgggatatgtctttggttgtcctgtgcgttctgtagttaacgagcgtgacgcggcccgtgtgaccgcccgcgacgggagagaatttgccgcgaaacggctggtctgcacgattcccttaaacgtacttagtaccatacaattctcgcctgccctgtctactgaacgtatcagtgctatgcaggctggtcatgtgaacatgtgtaccaaagtgcacgctgaggttgataataaggatatgcgttcttggtcggggatcgcatacccatttaataaactgtgtgtggcaataggagatggtactactccagcgggtaacacacatcttgtatgcttcggcacggatgcgaatcatattcagcccgatgaagacgttcgtgaaacattaaaggcggtaggccagttagcgcccgggacttttggggtaaaaagattggtgttccataattgggtcaaggacgaatttgccaaaggggcccgttttttctcccggcctgggatggtttccgagtgccttcagggactgcgtgaaaagcacagaggtgttgtgttcgccaactctgattgggctttagggtggcggagctttatagacggcgcaatagaggagggcacgagagcagctagagttgtgcttgaagaactggggactaaacgcgaagtgaaggcacggctgtga

SEQ ID NO: 4 - DNA, 1488 bp, Artificial Sequence
  Nucleotide sequence encoding amine oxidase mutant 2
atgaccagccgcgatggctatcagtggaccccggaaaccggcctgacccagggcgtgccgagcctgggcgtgattagcccgccgaccaacattgaagataccgataaagatggcccgtgggatgtgattgtgattggcggcggctattgcggcctgaccgcgacccgcgatctgaccgtggcgggctttaaaaccctgctgctggaagcgcgcgatcgcattggcggccgcagctggagcagcaacattgatggctatccgtatgaaatgggcggcacctgggtgcattggcatcagagccatgtgtggcgcgaaattacccgctataaaatgcataacgcgctgagcccgagctttaactttagccgcggcgtgaaccattttcagctgcgcaccaacccgaccaccagcacctatatgacccatgaagcggaagatgaactgctgcgcagcgcgctgcataaatttaccaacgtggatggcaccaacggccgcaccgtgctgccgtttccgcatgatatgttttatgtgccggaatttcgcaaatatgatgaaatgagctatagcgaacgcattgatcagattcgcgatgaactgagcctgaacgaacgcagcagcctggaagcgatgattctgtgctgcagcggcggcaccctggaaaacagcagctttggcgaatttctgcattggtgggcgatgagcggctatacctatcagggctgcgtggattgcctgaccagctataaatttaaagatggccagagcgcgtttgcgaaaaaattttgggaagaagcggcgggcaccggccgcctgggctatgtgtttggctgcccggtgcgcagcgtggtgaacgaacgcgatgcggcgcgcgtgaccgcgcgcgatggccgcgaatttgcggcgaaacgcctggtgtgcaccattccgctgaacgtgctgagcaccattcagtttagcccggcgctgagcaccgaacgcattagcgcgatgcaggcgggccatgtgagcatgtgcaccaaagtgcatgcggaagtggataacaaagatatgcgcagctggaccggcattgcgtatccgtttaacaaactgtgctatgcgattggcgatggcaccaccccggcgggcaacacccatctggtgtgctttggcaacagcgcgaaccatattcagccggatgaagatgtgcgcgaaaccctgaaagcggtgggccagctggcgccgggcacctttggcgtgaaacgcctggtgtttcataactgggtgaaagatgaatttgcgaaaggcgcgggcttttttagccgcccgggcatggtgagcgaatgcctgcagggcctgcgcgaaaaacatcgcggcgtggtgtttgcgaacagcgattgggcgctgggctggcgcagctttattgatggcgcgattgaagaaggcacccgcgcggcgcgcgtggtgctggaagaactgggcaccaaacgcgaagtgaaagcgcgcctgtga

SEQ ID NO: 5 - protein, 407 aa, Artificial Sequence
  Nicotine synthetase mutant
MASMTGGQQMGRGSEFHKMGNGKQALKSQRSEGPALVLAIGTATPSHWIDQSSYPDYYFRVTNSDHLVDLKEKFRRICSRTMIKKRHMLLTEEILKKNPNLCSFSEPSLDITFDILVSEIPKLGKEAALKAIQEWAQPKSTITHLVFCTRSGVDMPGADYQAIKLLGLGPSVQRLMMYQEGCAAGGTMLRLAKDLAENNKGARILVICAESKAIGFRGPSESHVDNLVPQALFGDGAAAIIVGSNPKLGLEKPRFEIVSAHQTFVPNGDCHLALHLREMGLTFHCTKDVPPTIAKNVESCLTKALEPLGISDWNSLFWILHPGGNAIVDQVENKLGLEHEVLRATTNILRDFGNMSSACVLFILDEIRKKSARDGLKTTGEGLDFGVLLSFGPTLTIETVVLHSKPI

SEQ ID NO: 6 - DNA, 1224 bp, Artificial Sequence
  Nucleotide sequence encoding nicotine synthetase mutant
atggcgagcatgaccggcggccagcagatgggccgcggcagcgaatttcataaaatgggcaacggcaaacaggcgctgaaaagccagcgcagcgaaggcccggcgctggtgctggcgattggcaccgcgaccccgagccattggattgatcagagcagctatccggattattattttcgcgtgaccaacagcgatcatctggtggatctgaaagaaaaatttcgccgcatttgcagccgcaccatgattaaaaaacgccatatgctgctgaccgaagaaattctgaaaaaaaacccgaacctgtgcagctttagcgaaccgagcctggatattacctttgatattctggtgagcgaaattccgaaactgggcaaagaagcggcgctgaaagcgattcaggaatgggcgcagccgaaaagcaccattacccatctggtgttttgcacccgcagcggcgtggatatgccgggcgcggattatcaggcgattaaactgctgggcctgggcccgagcgtgcagcgcctgatgatgtatcaggaaggctgcgcggcgggcggcaccatgctgcgcctggcgaaagatctggcggaaaacaacaaaggcgcgcgcattctggtgatttgcgcggaaagcaaagcgattggctttcgcggcccgagcgaaagccatgtggataacctggtgccgcaggcgctgtttggcgatggcgcggcggcgattattgtgggcagcaacccgaaactgggcctggaaaaaccgcgctttgaaattgtgagcgcgcatcagacctttgtgccgaacggcgattgccatctggcgctgcatctgcgcgaaatgggcctgacctttcattgcaccaaagatgtgccgccgaccattgcgaaaaacgtggaaagctgcctgaccaaagcgctggaaccgctgggcattagcgattggaacagcctgttttggattctgcatccgggcggcaacgcgattgtggatcaggtggaaaacaaactgggcctggaacatgaagtgctgcgcgcgaccaccaacattctgcgcgattttggcaacatgagcagcgcgtgcgtgctgtttattctggatgaaattcgcaaaaaaagcgcgcgcgatggcctgaaaaccaccggcgaaggcctggattttggcgtgctgctgagctttggcccgaccctgaccattgaaaccgtggtgctgcatagcaaaccgatttga

SEQ ID NO: 7 - protein, 336 aa, Artificial Sequence
  Phosphite dehydrogenase mutant
MLPKLVITHRVHDEILQLLAPHCELMTNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHERKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 8 - DNA, 1011 bp, Artificial Sequence
  Nucleotide sequence encoding phosphite dehydrogenase mutant
atgttaccgaaattagttatcacgcacagagtgcacgacgaaatccttcaattgctggcccctcattgtgagttgatgaccaaccaaaccgattctaccctgacgagagaagagatactgcgccgttgcagagacgcacaagccatgatggcgtttatgccggaccgtgtagatgcagactttcttcaagcttgcccggaacttcgggtcattggttgtgctttgaaagggttcgacaactttgacgtggatgcgtgtactgcacgcggggtatggcttacttttgtacctgacttattgacggttcccactgccgagcttgctattggcctggccgtcggattaggccgccatttacgtgcggcagatgcgttcgtacggagtgggaagtttcggggctggcaaccgcgattctacgggactggattggataacgccactgtaggtttccttgggatgggtgccataggtttagctatggcagatagattacaggggtggggagctacccttcaatatcatgagcgtaaagcattggatacacaaacagaacagcgcttgggtcttagacaggtcgcgtgctcggaacttttcgcttcctcagacttcatactgttggccttgccacttaacgctgacactctacatttggtaaacgctgaattgctggctttggtacgtcccggcgcactgttagttaatccgtgccggggctcggtggtagacgaggcagccgtgctggcagcgcttgagagagggcaacttggcggatatgctgcagacgtgttcgagatggaagactgggcccgcgcggaccgtccacagcaaatcgatcctgcgttgttggcccaccctaatactttatttactccgcacatcggatcagcggtgagagcggtgcggcttgagattgagcgttgcgcagctcagaacatcctccaggcgctggcaggagaacgtccaattaatgctgtaaatcgtttaccgaaggctgaaccagcagcttgttga

(19) United States
(12) Patent Application Publication
(10) Pub. No.: US 2024/0263204 A1
(43) Pub. Date: Aug. 8, 2024

(54) METHOD FOR PREPARING S-NICOTINE

(71) Applicant: SHENZHEN READLINE BIOTEC CO., LTD., Shenzhen, Guangdong (CN)

(72) Inventors: Hong ZHAO, Shenzhen, Guangdong (CN); Ruimei LING, Shenzhen, Guangdong (CN); Lin FU, Shenzhen, Guangdong (CN); Tiemei YU, Shenzhen, Guangdong (CN); Junfeng PAN, Shenzhen, Guangdong (CN); Jain LIU, Shenzhen, Guangdong (CN)

(73) Assignee: SHENZHEN READLINE BIOTEC CO., LTD., Shenzhen, Guangdong (CN)

(21) Appl. No.: 18/293,784

(22) PCT Filed: Oct. 11, 2021
(86) PCT No.: PCT/CN2021/123012

§ 371 Date: Jan. 31, 2024

(30) Foreign Application Priority Data
Aug. 10, 2021 (CN) 202110914222.4

(51) Int. Cl. C12P 17/16; C12N 9/02; C12N 9/06; C12N 9/08

(52) U.S. Cl. CPC C12P 17/165; C12N 9/0004; C12N 9/0022; C12N 9/0065; C12Y 104/03004; C12Y 111/01006; C12Y 120/01001

(57) ABSTRACT

The present invention relates to the technical field of biosynthesis, and in particular to a method for preparing S-nicotine. Amine oxidase is utilized to oxidize 1-methylpyrrolidine into corresponding imine, and then the imine and nicotinic acid are condensed and decarboxylated under the catalysis of nicotine synthetase to obtain a final product S-nicotine. The S-nicotine having specific chirality can be obtained by means of two-step reaction in a reaction system, the synthetic route is short, the yield is high, the reaction conditions are mild, and large-scale production is easy to achieve; moreover, raw materials are wide in source, low in price, low in production cost and environmentally friendly, the production cost of nicotine is remarkably reduced, and the requirements of current green industrial production can be better satisfied.

### CROSS REFERENCE TO RELATED APPLICATIONS

This application claims the priority of Chinese Patent Application No. 202110914222.4, filed with the China National Intellectual Property Administration on Aug. 10, 2021, and titled with “METHOD FOR PREPARING S-NICOTINE”, which is hereby incorporated by reference in its entirety.

### FIELD

The present disclosure relates to the field of biosynthesis, and specifically relates to a method for preparing S-nicotine.

### BACKGROUND

Nicotine is an important ingredient of tobacco, as well as a core raw material in e-cigarette formulations and the synthesis of certain nicotinic drugs.

Traditional methods for cultivating tobacco, extracting and purifying nicotine occupy a large area of land and take a long period of time. In addition, some highly toxic ingredients inevitably present in the products during the process of extraction, which are great harmful to the human body. Therefore, the direct synthesis of S-nicotine by chemical or biological processes has become an important approach for the preparation of S-nicotine.

Several common methods for preparing S-nicotine are as follows:

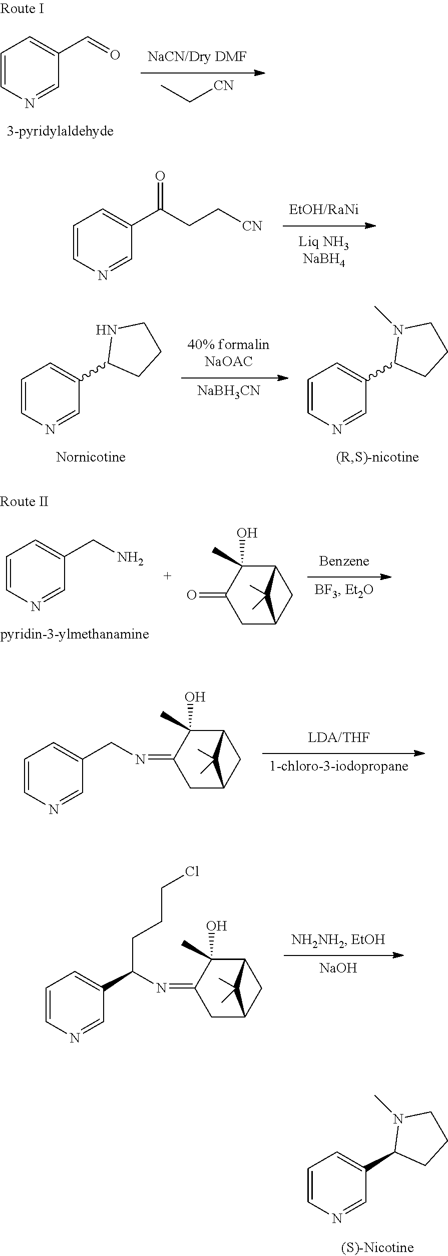

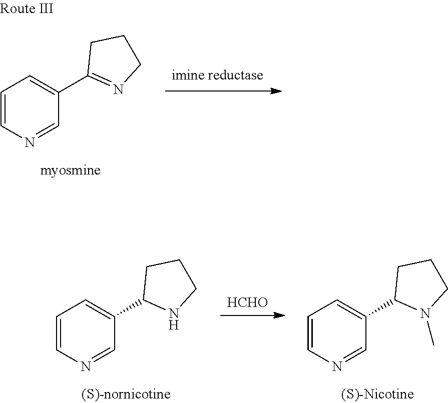

Route I shows a chemical method for preparing racemic nicotine. Pyridine acetaldehyde is used as raw material to prepare nicotine racemate through three-step chemical reaction, and then the nicotine racemate is subjected to chiral resolution by using chemical agents or enzymes to obtain S-nicotine. The above chemical reactions require complex and dangerous production process in which strong poisons (NaCN, etc.) and explosive products (e.g. Raney Ni for hydrogenation) are used. (Reference could be made to, International patent WO2017/119003 A1, “A PROCESS FOR THE PREPARATION OF NICOTINE”).

Route II shows a chemical method for directly preparing S-nicotine. Pyridine ethylamine is used as the starting material to obtain S-nicotine through three-step chemical conversion. The catalysts used in the first two steps are expensive and the reaction conditions are harsh; and the final yield is also low (<50%). (Reference could be made to, Josha T. Ayers, AAPS 2005, “A General Procedure for the Enantioselective Synthesis of the Minor Tobacco Alkaloids Nornicotine, Anabasine, and Anatabine”).

In route III, myosmine, a nicotine precursor, is used as raw material, which is subjected to chiral reduction using enzymes and to methylation using chemical agents to prepare S-nicotine. Although this route is relatively short and has a high yield, the production cost is high due to the use of expensive myosmine as a starting material.

It can be concluded that, among the above three classical preparation processes of S-nicotine, although the two chemical preparation methods shown in Route I and Route II use relatively cheap raw materials and the overall route is not long (three-step or four-step reaction), the chemical catalysts (BF₃, LDA, NaBH₄) used in the reaction are expensive, the operation of LDA and NaCN is dangerous, and so on, all lead to high environmental and safety costs in scale production. In Route III, myosmine is used as raw material to produce S-nornicotine through chiral reduction by using imine reductase, and then methylation is performed by chemical method. The drawback of this route is that it needs to use expensive myosmine as a raw material, thus greatly increasing the overall production cost. Also, with the development of science and technology, the country has an increasing high demand for a high green production index of chemical industry. Therefore, it is of great significance to provide a production process of S-nicotine with simple operation steps, low cost, safety and environmental protection.

### SUMMARY

In view of this, the present disclosure provides a method for producing S-nicotine, wherein 1-methylpyrrolidine and nicotinic acid are used as raw materials and converted to S-nicotine in one-time, with a simple process, high reaction yield, low cost and environmental friendliness.

In order to achieve the above object, the present disclosure provides the following technical solutions.

The present disclosure provides an amine oxidase mutant, wherein an amino acid sequence of the amine oxidase mutant is selected from the group consisting of:
amino acid sequence set forth in SEQ ID NO: 1 or SEQ ID NO: 2;
amino acid sequence derived from SEQ ID NO: 1 or SEQ ID NO: 2 by substitution, deletion or addition of one or more amino acids and functionally identical or similar to SEQ ID NO: 1 and SEQ ID NO: 2, and
amino acid sequence having at least 90% homology to the amino acid sequence set forth in SEQ ID NO: 1 or SEQ ID NO: 2 and functionally identical or similar to amino acid sequence set forth in SEQ ID NO: 1 or SEQ ID NO: 2.

In the present disclosure, amine oxidase mutant 1 (AO1) and amine oxidase mutant 2 (AO2) are both derived from a monoamine oxidase found in Aspergillus niger, and the amino acid sequence ID number of this wild-type enzyme is Uniprot ID: P46882, EC 1.4.3.4.

Wherein, the amine oxidase mutant comprising five point mutations of M242R, W230I, T354S, Y365V and W430R is named amine oxidase mutant 1 (abbreviated as AO1), of which the amino acid sequence is set forth in SEQ ID NO: 1.

In some embodiments, the amine oxidase mutant comprising 10 point mutations of F210M, L213C, M242V, 1246T, R259K, R260K, N336S, T384N, D385S and W430G, is named amine oxidase mutant 2 (abbreviated as AO2), of which the amino acid sequence is set forth in SEQ ID NO: 2.

The present disclosure further provides a nucleic acid encoding the amine oxidase mutants.

In some embodiments, a nucleotide sequence encoding the amine oxidase mutant is set forth in SEQ ID NO: 3 or SEQ ID NO: 4, wherein the nucleotide sequence encoding the amine oxidase mutant 1 (AO1) is set forth in SEQ ID NO: 3, and the nucleotide sequence encoding the amine oxidase mutant 2 (AO2) is set forth in SEQ ID NO: 4.

The present disclosure provides a nicotine synthetase mutant, wherein an amino acid sequence of the nicotine synthetase mutant is selected from the group consisting of:
amino acid sequence set forth in SEQ ID NO: 3,
amino acid sequence derived from SEQ ID NO: 5 by substitution, deletion or addition of one or more amino acids and functionally identical or similar to SEQ ID NO: 5, and
amino acid sequence having at least 90% homology to the amino acid sequence as shown in SEQ ID NO: 5 and functionally identical or similar to SEQ ID NO: 5.

In the present disclosure, the nicotine synthetase mutant is derived from an enzyme for oxidization-reduction condensation in Anisodus acutangulus, a solanaceous plant, and the amino acid sequence number of the wild-type nicotine synthetase (i.e. the enzyme for oxidization-reduction condensation) is 6J1M.

In some embodiments, the nicotine synthetase mutant comprises 14 point mutations of M17H, R112T, Q113F, L162A, Q180E, F183A, S212K, A229P, P248L, V254R, A261H, K341V, R346T and G394T, and the amino acid sequence of the nicotine synthetase mutant is set forth in SEQ ID NO: 5.

The present disclosure further provides a nucleic acid encoding the nicotine synthetase mutant.

In some embodiments, the nucleotide sequence encoding the nicotine synthetase mutant is set forth in SEQ ID NO: 6.

The present disclosure provides a phosphite dehydrogenase (PTDH) mutant and the amino acid sequence of the phosphite dehydrogenase mutant is selected from the group consisting of:
amino acid sequence set forth in SEQ ID NO: 7,
amino acid sequence derived from SEQ ID NO: 7 by substitution, deletion or addition of one or more amino acids and functionally identical or similar to SEQ ID NO: 7, and
amino acid sequence having at least 90% homology to the amino acid sequence set forth in in SEQ ID NO: 7 and functionally identical or similar to SEQ ID NO: 7.

In the present disclosure, the phosphite dehydrogenase (PTDH) mutant is modified from a wild-type phosphite dehydrogenase of Pseudomonas stutzeri, and the amino acid sequence number of the wild-type is Uniprot ID: 069054, EC 1.20.1.1.

In some embodiments, the phosphite dehydrogenase mutant comprises 13 point mutations of v71I, Q132R, E130K, Q137R, 1150F, A176R, Q215L, R275Q, L276Q, 1313L, V315A, A319E and A325V, of which the amino acid sequence is set forth in SEQ ID NO: 7.

The present disclosure further provides a nucleic acid encoding the phosphite dehydrogenase mutant.

In some embodiments, the nucleotide sequence encoding the phosphite dehydrogenase mutant is set forth in SEQ ID NO: 8.

The present disclosure provides an enzyme composition, comprising at least two enzymes selected from the group consisting of the following (a)-(b):
(a) an amine oxidase or mutant thereof;
(b) a nicotine synthetase or mutant thereof;
(c) a phosphite dehydrogenase or mutant thereof; and
(d) a catalase or mutant thereof.

In some embodiments of the present disclosure, the enzyme composition comprises two enzymes of (a) and (b), that is, the amine oxidase mutant and the phosphite dehydrogenase mutant.

In some embodiments, the enzyme composition of the present disclosure comprises the following (a)-(b):
(a) an amine oxidase or mutant thereof;
(b) a nicotine synthetase or mutant thereof;
(c) a phosphite dehydrogenase or mutant thereof; and
(d) a catalase or mutant thereof.

In some specific embodiments, the enzyme composition provided by the present disclosure comprises an amine oxidase mutant, a phosphite dehydrogenase mutant, a nicotine synthase mutant and a catalase.

In some embodiments, the present disclosure provides the above enzyme composition, wherein the amino acid sequence of the amine oxidase mutant is set forth in SEQ ID NO: 1 or SEQ ID NO: 2;
the amino acid sequence of the nicotine synthetase mutant is set forth in SEQ ID NO: 5;
the amino acid sequence of the phosphite dehydrogenase mutant is set forth in SEQ ID NO: 7; and
the catalase is obtained by purchase. In specific embodiments of the present disclosure, the catalase (Terminox Ultra) is purchased from Novozymes Enzymes.

The present disclosure further provides use of the enzyme composition in the preparation of S-nicotine.

The present disclosure further provides a method for producing S-nicotine, comprising,
under a condition of presence of solvent, oxygen and NADPH, mixing 1-methylpyrrolidine and nicotinic acid with the enzyme composition, performing reaction and obtaining S-nicotine.

In the preparation method provided by the present disclosure, 1-methylpyrrolidine is oxidized into corresponding imine using the amine oxidase or mutant thereof in the enzyme composition, and then the imine and nicotinic acid are condensed and decarboxylated under the catalysis of nicotine synthetase or mutant thereof to obtain S-nicotine. The synthesis route is shown in FIG. 1.

Wherein, oxygen is required in the first step of the oxidization reaction and the reaction produces a by-product of hydrogen peroxide. Therefore, in some embodiments of the present disclosure, hydrogen peroxide is effectively removed from the system by adding a small amount of catalase and O2 can also be recycled.

The coenzyme NADPH (nicotinamide adenine dinucleotide phosphate hydrogen) is required in the second step of the condensation and decarboxylation reaction. This coenzyme is relatively expensive and can be effectively regenerated by adding a NADPH regeneration system (phosphite dehydrogenase, PTDH) to the same system to greatly reduce its dosage and reduce the cost of production.

In some embodiments, the NADPH is produced by a NADPH regeneration system, and the NADPH regeneration system comprises beta-nicotinamide adenine dinucleotide phosphate monosodium salt, sodium phosphite pentahydrate and phosphite dehydrogenase mutant.

In some embodiments, the solvent is a solution of trihydroxymethylaminomethane hydrochloric acid or a solution of trihydroxymethylaminomethane hydrochloric acid containing a co-solvent. A co-solvent can promote the dissolution of each substrate in the solvent, which is conducive to the reaction. In the present disclosure, all kinds of common and feasible co-solvents are practicable, including but not limited to isopropanol, acetone and DMSO. In a specific embodiment of the present disclosure, isopropanol is used as the co-solvent for substrates, which is more effective.

A method for producing S-nicotine provided by the present disclosure comprises
adding 1-methylpyrrolidine nicotinic acid, beta-nicotinamide adenine dinucleotide phosphate monosodium salt, sodium phosphite pentahydrate and isopropanol into a solution of trihydroxymethylaminomethane hydrochloric acid, adjusting the pH to 6.5-9.0, and adding the enzyme composition to obtain a reaction system; and
stirring the reaction system for 4-8 hours at 25-35° C. at the oxygen pressure of 1.0-2.0 atmospheric pressure, adjusting the pH to 9.0-11.0 after the reaction, extracting with ethyl acetate, combining organic phases, drying, filtering and concentrating to obtain S-nicotine.

In a specific embodiment, a method for producing S-nicotine comprises
sequentially adding 1-methylpyrrolidine nicotinic acid, beta-nicotinamide adenine dinucleotide phosphate monosodium salt, sodium phosphite pentahydrate and isopropanol into a solution of trihydroxymethylaminomethane hydrochloric acid, after adjusting the pH to 8.0, adding the enzyme composition to obtain a reaction system; and
slowly stirring the reaction system for 6 hours at 30° C. at the oxygen pressure of 1.5 atmospheric pressure, adjusting the pH to 10.0 after the reaction, sequentially extracting with ethyl acetate, combining organic phases, drying, filtering and concentrating to obtain S-nicotine.

In some embodiments, the enzyme composition of the present disclosure comprises an amine oxidase mutant, a phosphite dehydrogenase mutant, a nicotine synthetase mutant and a catalase, wherein a ratio of enzyme activities of the amine oxidase mutant, the nicotine synthetase mutant, the phosphite dehydrogenase mutant and the catalase is preferably (1.5-2.5):(2.5-5):(4-8):1. In some specific embodiments, a ratio of enzyme activities of the amine oxidase mutant, the nicotine synthetase mutant, the phosphite dehydrogenase mutant and the catalase is 2:4:6:1.

In some embodiments, in the reaction system,
the concentration of the 1-methylpyrrolidine is 150-250 mM, and particularly may be 150 mM, 200 mM or 250 mM;
the concentration of the nicotinic acid is 100-300 mM; and particularly may be 150 mM, 200 mM or 250 mM;
the concentration of the beta-nicotinamide adenine dinucleotide phosphate monosodium salt is 0.2-0.6 mM, and particularly may be 0.2 mM, 0.4 mM or 0.6 mM;
the concentration of the sodium phosphite pentahydrate is 200-300 mM, and particularly may be 200 mM, 240 mM or 300 mM; and
the volume fraction of the isopropanol is 1-5%, and particularly may be 1% or 5%.

The present disclosure pioneers the use of amine oxidase to oxidize 1-methylpyrrolidine to the corresponding imine, and then this imine and nicotinic acid are condensed and decarboxylated under the catalysis of nicotine synthetase to produce the final product S-nicotine. By this method, the S-nicotine with specific chirality can be obtained by a two-step reaction in one reaction system, the synthetic route is short, the yield is high, the reaction conditions are mild, and large-scale production is easy. Moreover, the raw materials are widely available, low price, low production cost and environmentally friendly. The production cost of nicotine is remarkably reduced, while makes it more in line with the requirements of current green industrial production.

### BRIEF DESCRIPTION OF DRAWINGS

FIG. 1 shows the synthetic route of S-nicotine of the present disclosure.

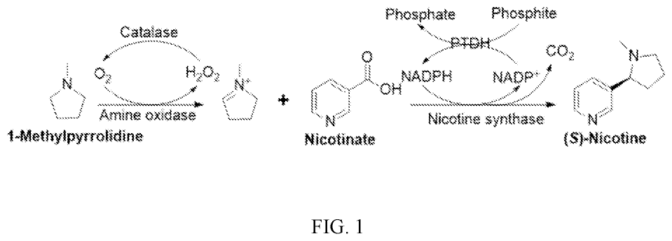

FIG. 2 shows the mass spectrogram of S-nicotine in Example 1 of the present disclosure.

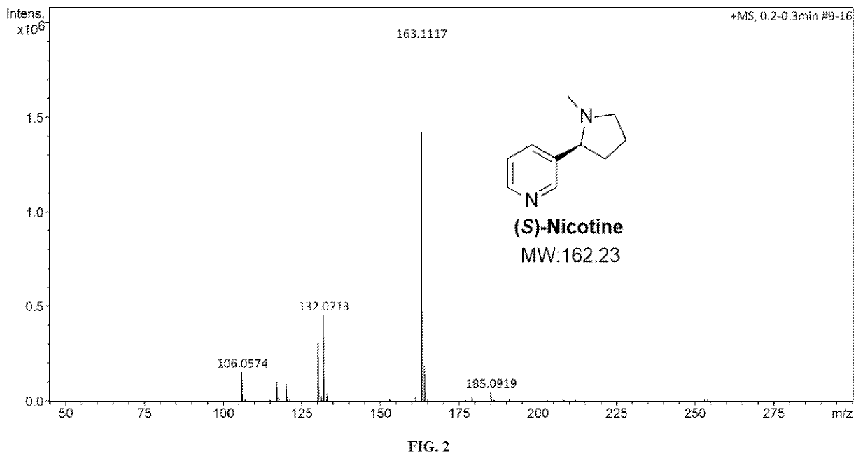

FIG. 3 shows the 1H-NMR spectrum of S-nicotine in Example 1 of the present disclosure, 400M Varian NMR, D₂O as a solvent.

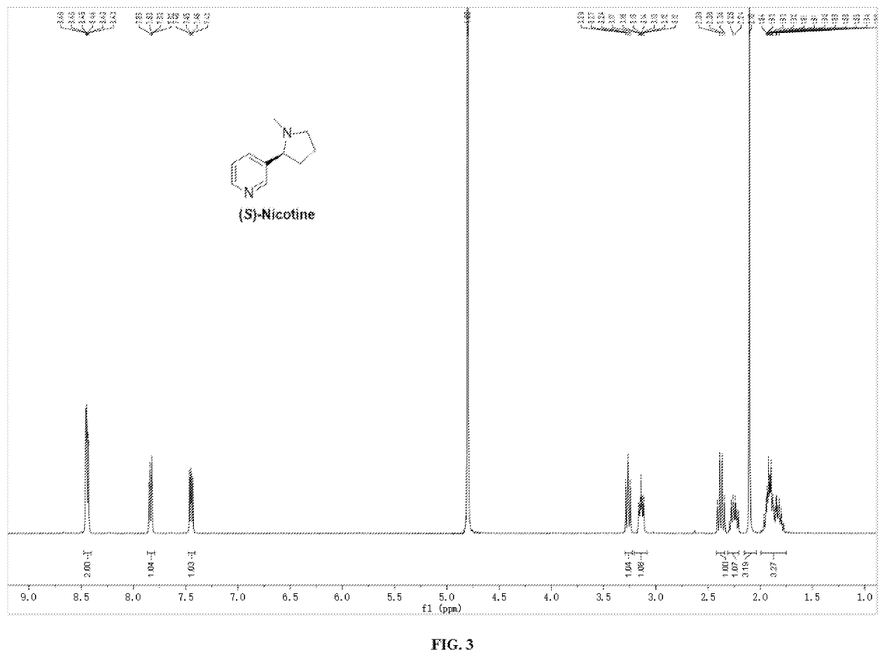

### DETAILED DESCRIPTION

The present disclosure provides a method for producing S-nicotine. Those skilled in this field may use the contents herein for reference and improve the process parameters appropriately to achieve. In particular, it should be noted that all similar replacements and modifications are apparent to those skilled in the art, which are all considered to be included in the present disclosure. The method and use of the present disclosure have been described through preferred embodiments, and those skilled in the art can apparently make modifications or appropriate changes and combinations to the method and use herein without departing from the content, spirit and scope of the present disclosure to realize and apply the technology of the present disclosure.

Unless otherwise specified, the reagents and materials used in the present disclosure are all common commercial products and available in the market.

In the present disclosure, the catalase (Terminox Ultra) was purchased from Novozymes. Other enzymes, the amine oxidase mutant 1 (AO1) set forth in SEQ ID NO: 1, the amine oxidase mutant 2 (AO2) set forth in SEQ ID NO: 2, the nicotine synthetase (NS) mutant set forth in SEQ ID NO: 5, and the phosphite dehydrogenase (PTDH) mutant set forth in SEQ ID NO: 7 were all obtained by constructing engineered strains and performing fermentation. The method is as follows.

The genes corresponding to the above enzyme mutants were synthesized by Anhui Tongyong Biology, and then subcloned into pET28a plasmid between NdeI/XhoI restriction sites. The constructed plasmids were transformed into E. coli strain BL21 (Tsingke Biology) and plate culture was carried out. The single clones were picked for stepwise liquid culture. Namely, a single colony was firstly cultured in 5 ml of LB medium containing 50 μM kanamycin at 37° C., and then inoculated to 250 ml of LB medium containing the same antibiotic after cells grew to logarithmic phase, and finally transferred to a 5 L culture fermenter for culture. When the OD value of cells was about 15, 0.5 mM isopropyl-βeta-D-thiogalactopyranoside (IPTG) was added to induce protein expression for 10 hours at 30° C., and then the cells were collected by high-speed centrifugation at 6000 rpm for 15 minutes to obtain 40-60 g of wet cells. A small amount cells from the wet cells was taken out and mixed well with a buffer solution of 50 mM trihydroxymethylaminomethane hydrochloric acid (Tris-HCl) with a pH of 8.0, and then the cells were broken by freeze-thawing. After high-speech centrifugation, the supernatant was used to determine the protein expression by sodium dodecyl sulfate-polyacrylamide gel electrophoresis (SDS-PAGE). The remaining wet cells with correct protein expression were also mixed well with the buffer solution on ice at about 200 ml of buffer solution per 10 g of wet cells, and then the cells were broken under high pressure, and the enzyme-containing supernatant was collected after high-speed centrifugation (16000 rpm, 45 min) for direct use or further purified and immobilized for use in a reaction using solid enzyme. For liquid enzyme reactions, the enzyme activity is in the range of 200-350 U/ml, and U indicates the amount of enzyme required to convert 1 μmol of substrate in one minute at room temperature. LB medium is composed of 1% tryptone, 0.5% yeast powder, 1% NaCl, 1% dipotassium hydrogen phosphate, 1% dipotassium hydrogen phosphate and 5% glycerol.

In the present disclosure, the enzyme composition may be in a liquid form and may also be in a solid form of immobilized enzymes. The immobilized enzymes can be recycled after the reaction and reused. In some embodiments, a liquid enzyme composition is used to prepare S-nicotine. In some other embodiments, an immobilized enzyme composition is used to prepare S-nicotine. The immobilized enzyme composition could be prepared according to the following steps:

Solid ammonium sulfate was added gradually to the crude solution of amine oxidase (AO1 or AO2), the crude solution of nicotine synthetase (NS) or the crude solution of phosphite dehydrogenase (PTDH) obtained by fermentation until solid enzymes were precipitated. The ratio of the weight of sodium sulfate to the volume of the solution was 25%-60% (w/v). The solid enzyme was subsequently collected by centrifugation (10,000 rpm, 12 minutes) and slowly dissolved in 25 mM Tris-buffer solution whit a pH of 8.0, and then desalted through a G25 size-exclusion chromatography column purchased from Sigma and separated using a DEAE Seplite FF anion-exchange column (Xi′an Lanxiao) to obtain the initially purified enzyme solutions of AO1, AO2, NS and PTDH. Finally, AO1 or AO2, NS, PTDH and catalase of Novozymes were mixed according to the ratio of the active unit of 2:4:6:1 and immobilized at one time by using LX-1000EP epoxy resin (Xi′an Lanxiao). The immobilization method is as follows: 1,000 U of mixed enzyme was dissolved in 1 L of 50 mM potassium phosphate solution with a pH of 8.0, and then 40 mM phenoxyacetic acid and 300 g of LX-1000 EP epoxy resin were added, stirred for 4 hours at room temperature, and then filtered to obtain the immobilized enzymes. The immobilized enzymes were washed three times each with clear water and 25 mM phosphate buffer solution with a pH of 8.0, and then dried at low temperature for later use. The activity of the immobilized enzyme composition is 65-92% of that of the corresponding liquid enzyme.

The present disclosure is further illustrated below in conjugated with examples.

### EXAMPLES

### Example 1 Preparation of S-Nicotine Using Liquid Enzymes (AO1 and NS) by One-Pot Synthesis

17 g of 200 mM 1-methylpyrrolidine, 24.6 g of 200 mM nicotinic acid, 3.0 g of 0.4 mM beta-nicotinamide adenine dinucleotide phosphate (NADP) monosodium salt, 52 g of 240 mM sodium phosphite pentahydrate and 100 ml of isopropanol (as a substrate co-solvent) were added successively to 1 L of 50 mM trihydroxymethylaminomethane hydrochloric acid (Tris. HCl) with a pH of 8.0. After the pH value was adjusted to 8.0 using NaOH aqueous solution, the enzyme composition was added to obtain a reaction solution, wherein, the enzyme composition consisted of 2000 U of AO1 (sequence set forth in SEQ ID NO: 1), 4000 U of NS (sequence set forth in SEQ ID NO: 5), 1000 U of catalase and 6000 U of PTDH (sequence set forth in SEQ ID NO: 7).

Subsequently, the reaction solution was transferred into a pressure-resistant reactor, and slowly stirred for 6 hours at 30° C. at the oxygen pressure of 1.5 atmospheric pressure. After the reaction, the pH of the solution was adjusted to 10, and the solution was extracted for three times with 700 ml of ethyl acetate. The extracted organic phases were combined, dried with anhydrous sodium sulfate, filtered and concentrated to obtain 22 grams of light yellow liquid with a yield of 68% and a purity of 91% measured by HPLC.

### Example 2 Preparation of S-Nicotine Using Liquid Enzymes (AO2 and NS) by One-Pot Synthesis

The difference from Example 1 is that AO1 was replaced with amino oxidase AO2, and the other processes are the same. Similarly, 17 g of 200 mM 1-methylpyrrolidine, 24.6 g of 200 mM nicotinic acid, 3.0 g of 0.4 mM beta-nicotinamide adenine dinucleotide phosphate (NADP) monosodium salt, 52 g of 240 mM sodium phosphite pentahydrate and 100 ml of isopropanol were added successively to 1 L of solution of 50 mM trihydroxymethylaminomethane hydrochloric acid with a pH of 8.0 (Tris. HCl). After the pH value was adjusted to 8.0 using NaOH aqueous solution, the enzyme composition was added to obtain a reaction solution, wherein, the enzyme composition consisted of 2000 U of AO2 (sequence set forth in SEQ ID NO: 2), 4000 U of NS (sequence set forth in SEQ ID NO: 5), 1000 U of catalase and 6000 U of PTDH (sequence set forth in SEQ ID NO: 7).

Subsequently, the reaction solution was transferred into a pressure-resistant reactor, and slowly stirred for 4 hours at 30° C. at the oxygen pressure of 1.5 atmospheric pressure maintained. After the completion of the reaction was determined by HPLC and the pH of the solution was adjusted to 10, and the solution was extracted for three times with 700 ml of ethyl acetate. The extracted organic phases were combined, dried with anhydrous sodium sulfate, filtered and concentrated to obtain 29.8 grams of light yellow liquid with a yield of 92%, and the purity of S-nicotine was 95% detected by chromatogram.

### Example 3 Preparation of S-Nicotine Using Immobilized Enzyme Composition (AO2, NS, PTDH and Catalase) by One-Pot Synthesis

Example 3 was similar to Example 2, and the difference was that an immobilized enzyme composition prepared by the method of the present disclosure was used. The immobilized enzyme could be recycled for use after reaction. Similarly, 8.5 g of 100 mM 1-methylpyrrolidine, 12.3 g of 100 mM nicotinic acid, 1.5 g of 0.2 mM beta-nicotinamide adenine dinucleotide phosphate (NADP⁺) monosodium salt, 26 g of 120 mM sodium phosphite pentahydrate and 100 ml of isopropanol were added to 1 L of solution of 50 mM trihydroxymethylaminomethane hydrochloric acid (Tris·HCl) with a pH of 8.0. After the pH value was adjusted to 8.0 using NaOH aqueous solution, 6000-8000 U of immobilized enzyme (i.e. enzyme composition) was added to obtain a reaction solution, wherein, the enzyme composition comprised the AO2 mutant (sequence set forth in SEQ ID NO: 2), the NS mutant (sequence set forth in SEQ ID NO: 5), the PTDH mutant (sequence set forth in SEQ ID NO: 7) and catalase.

Subsequently, the reaction solution was transferred into a pressure-resistant reactor, and slightly shaken for 12 hours at 30° C. at the oxygen pressure of 1.5 atmospheric pressure. After the reaction, the immobilized enzyme composition was filtered, washed with 50 mM Tris buffer solution with a pH of 8.0 for three times to be recycled and then restored at 4° C. for later use. After the pH was adjusted to 10, the filtrate was extracted for three times with 700 ml of ethyl acetate. The extracted organic phases were combined, dried with anhydrous sodium sulfate, filtered and concentrated to obtain 13.6 grams of light yellow liquid with a yield of 92% and a purity of 98%. The recycled immobilized enzyme composition after filtration maintained 75-90% of enzyme activity of the original enzymes.

### Comparative Example 1 (No Co-Solvent)

Similar to example 2, 8.5 g of 100 mM 1-methylpyrrolidine, 12.3 g of 100 mM nicotinic acid, 3.0 g of 0.4 mM beta-nicotinamide adenine dinucleotide phosphate (NADP) monosodium salt, and 26 g of 120 mM sodium phosphite pentahydrate were added successively to 1 L of solution of 50 mM trihydroxymethylaminomethane hydrochloric acid (Tris·HCl) with a pH of 8.0. After the pH value was adjusted to 8.0 using NaOH aqueous solution, the enzyme composition was added to obtain a reaction solution, wherein, the enzyme composition consisted of 1000 U of AO2 (sequence set forth in SEQ ID NO: 2), 2000 U of NS (sequence set forth in SEQ ID NO: 5), 3000 U of PTDH (sequence set forth in SEQ ID NO: 7) and 1000 U of catalase.

Subsequently, the reaction solution was transferred into a pressure-resistant reactor, and stirred for 8 hours at 30° C. at the oxygen pressure of 1.5 atmospheric pressure. After the completion of the reaction was determined by HPLC, the pH of the solution was adjusted to 10, and the solution was extracted for three times with 500 ml of ethyl acetate. The extracted organic phases were combined, dried with anhydrous sodium sulfate, filtered and concentrated to obtain 6.8 grams of light yellow liquid with a yield of 43%, and the purity of S-nicotine was 84% detected by chromatogram.

### Comparative Example 2 (Wild-Type AO)

Similar to the above Example 1, 8.5 g of 100 mM 1-methylpyrrolidine, 12.3 g of 100 mM nicotinic acid, 3.0 g of 0.4 mM disodium beta-nicotinamide adenine dinucleotide phosphate (NADP) monosodium salt, 26 g of 120 mM sodium phosphite pentahydrate and 100 ml of isopropanol as a substrate co-solvent were added successively to 1 L of solution of 50 mM trihydroxymethylaminomethane hydrochloric acid (Tris·HCl) with a pH of 8.0. After the pH value was adjusted to 8.0 using NaOH aqueous solution, the enzyme composition was added to obtain the reaction solution, wherein, the enzyme composition consists of 6000 U of wild-type AO (Uniprot ID: P46882, EC 1.4.3.4), 2000 U of NS (sequence set forth in SEQ ID NO: 5), 3000 U of PTDH (sequence set forth in SEQ ID NO: 7) and 1000 U of catalase.

Subsequently, the reaction solution was transferred into a pressure-resistant reactor, and slowly stirred for 12 hours at 30° C. at the oxygen pressure of 1.5 atmospheric pressure. After the reaction, the pH of the solution was adjusted to 10, and the solution was extracted for three times with 800 ml of ethyl acetate. The extracted organic phases were combined, dried with anhydrous sodium sulfate, filtered and concentrated to obtain 2.6 grams of light yellow liquid with a yield of 16% and a purity of 71% detected by HPLC.

The above embodiments are only preferred embodiments of the present disclosure. It should be noted that, for those skilled in the art, several improvements and modifications may be further made without departing from the principle of the present disclosure, and these improvements and modifications should also be deemed as falling into the protection scope of the present disclosure.

1. An amine oxidase mutant, wherein an amino acid sequence of the amine oxidase mutant is selected from the group consisting of:
amino acid sequence set forth in SEQ ID NO: 1 or SEQ ID NO: 2,
amino acid sequence derived from SEQ ID NO: 1 or SEQ ID NO: 2 by substitution, deletion or addition of one or more amino acids and functionally identical or similar to SEQ ID NO: 1 and SEQ ID NO: 2, and
amino acid sequence having at least 90% homology to the amino acid sequence set forth in SEQ ID NO: 1 or SEQ ID NO: 2 and functionally identical or similar to amino acid sequence set forth in SEQ ID NO: 1 or SEQ ID NO: 2.

2. A nucleic acid, encoding the amine oxidase mutant according to claim 1.

3. The nucleic acid according to claim 2, wherein the nucleotide sequence of the nucleic acid is set forth in SEQ ID NO: 3 or SEQ ID NO: 4.

4. A nicotine synthetase mutant, wherein an amino acid sequence of the nicotine synthetase mutant is selected from the group consisting of:
amino acid sequence set forth in SEQ ID NO: 3,
amino acid sequence derived from SEQ ID NO: 5 by substitution, deletion or addition of one or more amino acids and functionally identical or similar to SEQ ID NO: 5, and
amino acid sequence having at least 90% homology to the amino acid sequence as shown in SEQ ID NO: 5 and functionally identical or similar to SEQ ID NO: 5.

5. A nucleic acid, encoding the nicotine synthetase mutant according to claim 4.

6. The nucleic acid according to claim 5, wherein the nucleotide sequence of the nucleic acid is set forth in SEQ ID NO: 6.

7-9. (canceled)

10. An enzyme composition, comprising two enzymes of the following (a)-(b):
(a) an amine oxidase or mutant thereof; and
(b) a nicotine synthetase or mutant thereof.

11. The enzyme composition according to claim 10, comprising the amine oxidase mutant and the nicotine synthetase mutant;
wherein the amino acid sequence of the amine oxidase mutant is set forth in SEQ ID NO: 1 or SEQ ID NO: 2; and
the amino acid sequence of the nicotine synthetase mutant is set forth in SEQ ID NO: 5.

12. The enzyme composition according to claim 21,
wherein the enzyme composition comprises the phosphite dehydrogenase mutant and;
the amino acid sequence of the phosphite dehydrogenase mutant is set forth in SEQ ID NO: 7.

13. (canceled)

14. A method for producing S-nicotine, comprising, under a condition of presence of a solvent, oxygen and NADPH, mixing 1-methylpyrrolidine and nicotinic acid with the enzyme composition according to claim 10, performing reaction and obtaining S-nicotine.

15. The method according to claim 14, wherein the NADPH is produced by a NADPH regeneration system in the reaction, and the NADPH regeneration system comprises beta-nicotinamide adenine dinucleotide phosphate monosodium salt, sodium phosphite pentahydrate and a phosphite dehydrogenase.

16. The method according to claim 15, comprising:
adding 1-methylpyrrolidine nicotinic acid, beta-nicotinamide adenine dinucleotide phosphate monosodium salt, sodium phosphite pentahydrate and isopropanol into a solution of trihydroxymethylaminomethane hydrochloric acid, adjusting the pH to 6.5-9.0, and adding the enzyme composition to obtain a reaction system; and
stirring the reaction system for 4-8 hours at 25-35° C. at the oxygen pressure of 1.0-2.0 atmospheric pressure, adjusting the pH to 9.0-11.0 after the reaction, extracting with ethyl acetate, combining organic phases, drying, filtering and concentrating to obtain S-nicotine.

17. The method according to claim 14, wherein the enzyme composition comprises the amine oxidase mutant, the phosphite dehydrogenase mutant, the nicotine synthetase mutant and the catalase.

18. The method according to claim 17, wherein a ratio of enzyme activities of the amine oxidase mutant, the nicotine synthetase mutant, the phosphite dehydrogenase mutant and the catalase is (1.5-2.5):(3-5):(4-8):1.

19. The method according to claim 15, wherein in the reaction,
the concentration of the 1-methylpyrrolidine is 150-250 mM;
the concentration of the nicotinic acid is 100-300 mM;
the concentration of the beta nicotinamide adenine dinucleotide phosphate monosodium salt is 0.2-0.6 mM;
the concentration of the sodium phosphite pentahydrate is 200-300 mM; and
the volume fraction of the isopropanol is 1-5%.

20. The enzyme composition according to claim 10, further comprising an enzyme selected from the group consisting of a phosphite dehydrogenase or mutant thereof and a catalase or mutant thereof.

21. The enzyme composition according to claim 20, comprising the phosphite dehydrogenase or mutant thereof.

22. The enzyme composition according to claim 20, comprising the catalase or mutant thereof.

23. The method according to claim 14, wherein the solvent is trihydroxymethylaminomethane hydrochloric acid or trihydroxymethylaminomethane hydrochloric acid containing a co-solvent.
